(19) United States
(12) Patent Application Publication
(10) Pub. No.: US 2022/0362479 A1
(43) Pub. Date: Nov. 17, 2022

(54) DOSE SENSING MODULE

(71) Applicant: Novo Nordisk A/S, Bagsvaerd (DK)

(72) Inventor: Nikolaj Eusebius Jakobsen, Soeborg (DK)

(73) Assignee: Novo Nordisk A/S, Bagsvaerd (DK)

(21) Appl. No.: 17/767,144

(22) PCT Filed: Oct. 8, 2020
(86) PCT No.: PCT/EP2020/078274

§ 371 Date: Apr. 7, 2022

(30) Foreign Application Priority Data
Oct. 8, 2019 (EP) 19201824.0

(51) Int. Cl. A61M 5/315; A61M 5/31

(52) U.S. Cl. CPC A61M 5/31568; A61M 5/31583; A61M 5/3135; A61M 5/31515; A61M 2205/50; A61M 2205/3561

(57) ABSTRACT

The present invention provides a sensor module (50) adapted to be arranged in a cartridge based drug delivery device between a rotatable piston rod and a cartridge piston. The sensor module (50) comprises a first sensor structure (52, 152, 252, 352) adapted to be at least substantially rotationally locked with respect to the cartridge piston and comprising a transversal sensor surface (52.2, 152.2, 252.2, 352.2), and a second sensor structure (53, 153, 253, 353) adapted to be rotationally locked with respect to the piston rod and comprising one or more flexibly supported and axially deflectable contact members (53.1, 53.2, 153.2, 253.2, 353.1, 353.2), which are positioned distally of the transversal sensor surface (52.2, 152.2, 252.2, 352.2) and are adapted to apply a proximally directed force thereto. The first sensor structure (52, 152, 252, 352) and the second sensor structure (53, 153, 253, 353) are adapted to undergo relative rotational motion, whereby the one or more contact members (53.1, 53.2, 153.2, 253.2, 353.1, 353.2) sweep the transversal sensor surface (52.2, 152.2, 252.2, 352.2). A processor (52.5, 152.5, 252.5) determines a relative angular displacement between the first sensor structure (52, 152, 252, 352) and the second sensor structure (53, 153, 253, 353) from signals generated as the one or more contact members (53.1, 53.2, 153.2, 253.2, 353.1, 353.2) sweep the transversal sensor surface (52.2, 152.2, 252.2, 352.2).

### FIELD OF THE INVENTION

The present invention relates to rotary encoders for use in drug delivery devices and to drug delivery devices employing rotary encoders for automatically capturing an amount of drug expelled from a drug reservoir.

### BACKGROUND OF THE INVENTION

Injection devices, such as injection pens, are widely used for self-administration of liquid drugs by people in need of therapeutic treatment. Many injection devices are capable of repeatedly setting and injecting either a fixed or a variable volume of drug upon operation of respective dose setting and dose expelling mechanisms in the device. Some injection devices are adapted to be loaded with a prefilled drug reservoir containing a volume of drug which is sufficient to provide for a number of injectable doses. When the reservoir is empty, the user replaces it with a new one and the injection device can thus be used again and again. Other injection devices are prefilled when delivered to the user and can only be used until the drug reservoir has been emptied, after which the whole injection device is discarded. The various injection devices typically expel the drug by advancing a piston in the reservoir using a motion-controlled piston rod.

Within some therapy areas the tendency of a patient to adhere to the prescribed therapy is dependent on the simplicity of the specific treatment regimen. For example, many people with type 2 diabetes are diagnosed with the disease at a relatively high age where they are less prone to accept a treatment that intervenes too much with their normal way of living. Most of these people do not like to be constantly reminded of their disease and, as a consequence, they do not want to be entangled in complex treatment patterns or waste time on learning to operate cumbersome delivery systems. In essence, many are of the opinion that the less manual involvement the better.

For a person with diabetes it is important to timely administer one or more glucose regulating agents to maximise the time spent in normoglycemia. In that connection, in order to establish an overview of one's adherence to a particular treatment regimen, it is significant to keep track of both when such a regulating agent is administered and how much is administered. Accordingly, it is recommended that the person keeps a log of administered dose sizes and times of administration.

Previously, the establishment and maintenance of such a log would require manually noting down the data, e.g. on paper or a pc. However, as this would entail frequent active involvement many people neglected the importance of establishing the overview. In recognition of this undesirable situation various solutions have been suggested for automatic capturing of the relevant information from the individual injection devices.

For example, WO 2018/078178 (Novo Nordisk A/S) discloses a pen type injection device having a sensor arranged on a deflectable exterior surface of the injection device housing. The deflectable exterior surface is configured to undergo a deflection at a specific angular displacement of an interior component rotationally locked to the piston rod, and the sensor is adapted to output a signal in response to a detected deflection, the signal thus being representative of the angular displacement of the piston rod. Since the amount of drug expelled by the disclosed injection device correlates with the total angular displacement of the piston rod relative to the housing the output signals are automatically captured by a processor in the injection device and used as a basis for an estimation of the administered dose. In addition, the processor may establish a time for reception of the output signals and provide a time stamp for the dose expelling event. The data may then be retrieved via an electronic display on the injection device or by wireless transmission to an external device e.g. having, or being connectable to, a display.

An alternative dose detection solution is disclosed in WO 2018/141571 (Novo Nordisk A/S) which concerns a disposable pen-type drug delivery device with a fully integrated sensor unit in the form of a piston washer module arranged between the piston rod of the dose expelling mechanism and the cartridge piston. The sensor unit operates like a rotary encoder and comprises a first sensor part which is rotationally locked with respect to the piston rod and a second sensor part which is rotationally locked with respect to the cartridge piston. The relative angular displacement between the two sensor parts exhibited during a dose expelling event when the piston rod rotates relative to the drug delivery device housing and the cartridge is detected galvanically and translated to an estimate of the size of the administered dose. In WO 2020/011710 (Novo Nordisk A/S) the two sensor parts are accommodated in a two-part module housing.

Most of the sensor embodiments presented in WO 2018/141571 and WO 2020/011710 involve axially directed contacts and sensor surfaces. For these sensor types, the reliability of the signal output depends on the axial contact pressure in the physical interface between the two sensor parts. To obtain a satisfactory contact pressure while also taking account of the desire to minimise the torque transferred from the first sensor part to the second sensor part it is suggested that the contacts be elastically supported, e.g. arranged on axially flexible arms.

However, when the first sensor part is pressed axially against the second sensor part during a dose expelling event the flexible arms become deflected and store elastic energy which is not released until the end of the piston rod movement. This elastic energy may, when eventually released, cause an additional movement of the piston and thereby contribute to prolonging the dose expelling event. A prolonged dose expelling may be viewed as a nuisance, especially by people who are already reluctant to performing injections. Furthermore, there is a greater risk that a user will pull out the injection needle prematurely in the expectation that the expelling procedure is finalised, only to find that liquid is still pouring out of the needle end. This may cause confusion as to why the dose expelling mechanism has not stopped and whether the correct dose has been received.

### SUMMARY OF THE INVENTION

It is an object of the invention to eliminate or reduce at least one drawback of the prior art, or to provide a useful alternative to prior art solutions.

In particular, it is an object of the invention to provide a rotary encoder based dose sensing module for use in a drug delivery device to automatically capture information regarding a delivered dose, which dose sensing module provides stable output signals and has an acceptable internal torque transmission, yet does not negatively affect the dose expelling procedure.

It is a further object of the invention to provide a drug delivery device with such a dose sensing module.

In the disclosure of the present invention, aspects and embodiments will be described which will address one or more of the above objects and/or which will address objects apparent from the following text.

In one aspect the invention provides a sensor module as defined in claim 1.

Accordingly, a sensor module for use in a cartridge based drug delivery device, such as an injection device, e.g. of the pen-shaped type, is provided. The sensor module which extends along a reference axis from a proximal module portion to a distal module portion is adapted to be arranged in the drug delivery device between a rotatable piston rod and a cartridge piston such that the proximal module portion interfaces with the piston rod and the distal module portion interfaces with the cartridge piston.

The sensor module comprises a module housing and a rotary encoder system powered by a power source, such as e.g. a battery. The rotary encoder system comprises a first sensor structure adapted to be at least substantially rotationally locked with respect to the cartridge piston and comprising a transversal sensor surface axially restricted with respect to the module housing, i.e. being either axially fixed to or capable of limited axial motion relative to the module housing. The rotary encoder system further comprises a second sensor structure adapted to be rotationally locked with respect to the piston rod, and a processor. The second sensor structure comprises one or more, i.e. a single or a plurality of, flexibly supported and axially deflectable contact members.

The first sensor structure and the second sensor structure are capable of undergoing relative rotational motion about the reference axis, and the one or more contact members are adapted to sweep the transversal sensor surface in response to such a relative rotational motion, thereby generating a plurality of signals, e.g. electrical signals, indicative of a relative angular displacement between the first sensor structure and the second sensor structure.

The generated signals are picked up and used by the processor to determine a total relative angular displacement between the first sensor structure and the second sensor structure exhibited during a dose expelling event performed with or by the drug delivery device. Since during such a dose expelling event the first sensor structure is at least substantially rotationally locked with respect to the cartridge and the second sensor structure is rotationally locked with respect to the piston rod, the processor thus estimates a total relative angular displacement of the piston rod which is correlatable with the size of the dose expelled. A thus determined total relative angular displacement between the first sensor structure and the second sensor structure and/or estimated size of the dose expelled may be relayed to an external device, e.g. wirelessly using wireless transmission means in the sensor module. Alternatively, the processor may be electrically connectable to an electronic display on the drug delivery device for visual presentation of the estimated dose size.

Importantly, the one or more contact members are positioned distally of the transversal sensor surface and adapted to apply a proximally directed force thereto. Thereby, when the piston rod moves distally during a dose expelling action the contact pressure in the interface between the one or more contact members and the transversal sensor surface will not increase because the first sensor structure and the second sensor structure are not pushed together. Resultantly, elastic energy will not accumulate in the flexible support of the one or more contact members as the dose expelling takes place, so no subsequent relaxation of the flexible support will cause additional piston movement after the dose expelling mechanism has reached its end-of-dose position.

In an exemplary embodiment of the invention the transversal sensor surface comprises a plurality of electrically conductive sensor areas arranged in a pattern, and the one or more contact members are adapted to sweep at least a subset of the plurality of electrically conductive sensor areas as the first sensor structure and the second sensor structure undergo relative rotation, thereby alternately connecting and disconnecting different sensor areas, a current connection being indicative of a current relative angular position of the first sensor structure and the second sensor structure. Electrical signals are thus generated for immediate processing in the processor which ultimately calculates the total relative angular displacement between the first sensor structure and the second sensor structure from the connections made, and on the basis thereof calculates a corresponding dose size. Alternatively, the dose size may be calculated by an external device receiving data from the sensor module.

The transversal sensor surface may be a distal surface of a rigid support sheet which extends perpendicularly to the reference axis within the module housing. The rigid support sheet may be or comprise a printed circuit board, and it may further comprise a proximal surface carrying the processor and other electronic components such as a wireless transmitter or transceiver module. A perpendicular rigid support sheet carrying the transversal sensor enables a very compact sensor module with few internal components.

In exemplary embodiments of the invention the rigid support sheet has a central through-going bore, and the proximal module portion comprises an axial pin member extending through the through-going bore and comprising a proximal pin end portion and a distal pin end portion. The proximal pin end portion is configured for rotational interlocking engagement with a distal end portion of the piston rod and the distal pin end portion is rotationally interlocked with the second sensor structure. Hence, in use, a rotation of the piston rod is transferred to the axial pin member and further on to the one or more contact members that consequently sweep the transversal sensor surface.

The sensor module may further comprise anti-rotation means adapted to interface with an interior wall portion of a cartridge in the cartridge based drug delivery device to impede relative angular displacement between the module housing and the cartridge. Such relative angular displacement may otherwise potentially occur due to the torque exerted by the second sensor structure on the first sensor structure as the piston rod rotates and the one or more contact members slide along the transversal sensor surface. In exemplary embodiments the anti-rotation means comprises a plurality of protrusions evenly distributed along a circumference of the distal module portion.

The plurality of electrically conductive sensor areas may be arranged to form a first circular track and a second circular track, where the first circular track is a code track and the second circular track is a ground track, and the one or more contact members may constitute one or two code contact members adapted to sweep the first circular track and one ground contact member adapted to sweep the second circular track. An embodiment with two code contact members provides for a particularly robust design of the rotary encoder system, whereas an embodiment with only one code contact member provides for a simpler and less tolerance critical design.

In an exemplary embodiment the first circular track comprises 36 evenly distributed code fields, and the second sensor structure comprises two code contact members exhibiting a 45° angular separation.

In another exemplary embodiment the first circular track comprises 72 evenly distributed code fields, and the second sensor structure comprises a single code contact member.

The plurality of electrically conductive sensor areas may alternatively form a single circular track comprising alternating code fields and ground fields, e.g. 40 evenly distributed fields where every other field is a code field and every other field is a ground field, and the one or more contact members may constitute three contact members exhibiting a 120° angular separation from each other. This configuration eliminates the need for a separate ground track at a different location on the transversal sensor surface.

If the rotary encoder system is powered by a battery, said battery may be arranged in the module housing distally of the transversal sensor surface, and the distal pin end portion may comprise a contact surface which abuts the battery, connecting electrically to a negative battery terminal. Thereby, a secondary ground connection is provided which functions as a back-up ground connection to the primary ground connection in case the operation of the drug delivery device entails significant internal vibrations in the sensor module.

The axial pin/battery connection may, in the alternative, be a primary ground connection in the rotary encoder system, in which case the separate ground track can be avoided, and the plurality of electrically conductive sensor areas may be arranged to form a single circular track of code fields.

In another aspect the invention provides a drug delivery device comprising a housing accommodating a dose expelling mechanism comprising a rotatable piston rod, and a cartridge rotationally fixed with respect to the housing, the cartridge comprising a drug chamber, sealed distally by a self-sealing septum and proximally by a cartridge piston, wherein a sensor module as described above is arranged in the drug delivery device between the piston rod and the cartridge piston.

The sensor module may be arranged such that the proximal module portion is rotationally fixed to the piston rod and the distal module portion abuts the cartridge piston.

In particular embodiments of the invention the piston rod comprises a distal indentation, and the proximal pin end portion is friction fitted in the indentation. Depending on the exterior shape of the proximal pin end portion, e.g. being elliptic cylindrical or square cylindrical, this allows for a fitting which can be carried out in a plurality of relative angular orientations of the piston rod and the axial pin member, thereby making it easier to obtain a proper alignment of the two components during assembly of the drug delivery device.

If the proximal pin end portion is circular cylindrical it allows for an axisymmetric fitting which is independent of any specific relative angular orientation of the piston rod and the axial pin member and which thereby is very easy to carry out during assembly.

For the avoidance of any doubt, in the present context the term “drug” designates a medium which is used in the treatment, prevention or diagnosis of a condition, i.e. including a medium having a therapeutic or metabolic effect in the body. Further, the terms “distal” and “proximal” denote positions at or directions along a drug delivery device, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 shows a dose detection principle according to the prior art,

FIG. 2 is a perspective longitudinal section view of an injection device with an integrated dose sensing module according to an exemplary embodiment of the invention,

FIG. 3 is an exploded view of the dose sensing module,

FIG. 4 is a perspective longitudinal section view of the dose sensing module,

FIG. 5 is a side view of a wiper assembly used in the dose sensing module,

FIG. 6 is a distal perspective view of the wiper assembly, and

FIGS. 7-9 are respective examples of alternative wiper assemblies for use in the dose sensing module.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

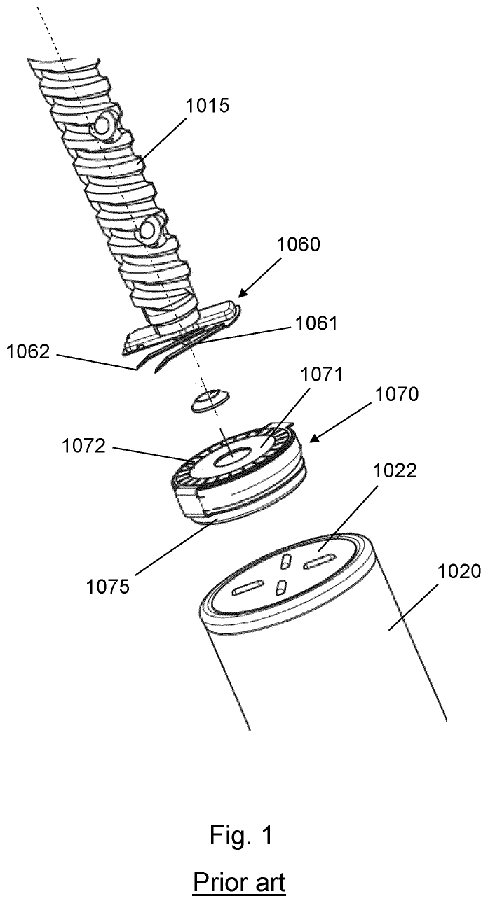

FIG. 1 shows a rotary sensor module according to the prior art, arranged between a distal end of a piston rod 1015 and a proximal end of a piston 1022 sealing a drug containing cartridge 1020. The sensor module, which is powered by a coin cell type battery 1075, comprises a first sensor part 1070 in the form of a flexible printed circuit board sheet having a proximally directed sensor surface 1071 on which 24 individual electrically conductive sensor areas 1072 are disposed circumferentially about a centre axis, and a second sensor part 1060 mounted on a distal end portion of the piston rod 1015 opposite the first sensor part 1070 and having contact structures in the form of two electrically connected flexible arms 1061, each terminating in a contact point 1062.

The first sensor part 1070 is adapted to engage, directly or indirectly, the piston 1022 such that no relative rotation therebetween is possible. The second sensor part 1060 is rotationally fixed to the piston rod 1015, and the contact points 1062 are adapted to engage and electrically connect various individual electrically conductive sensor areas 1072 upon relative rotational motion between the first sensor part 1070 and the second sensor part 1060, experienced as the piston rod 1015 rotates during a dose expelling action. This allows for an estimation of a total angular displacement exhibited by the piston rod 1015 during the dose expelling action and thereby of the amount of drug expelled.

During the dose expelling the piston rod 1015 undergoes a helical motion, and the axial component of this motion causes an axial advancement of the piston 1022 in the cartridge 1020, as the axial force from the piston rod 1015 is transferred to the proximal surface of the piston 1022 via the sensor module. In connection therewith the second sensor part 1060 is pressed against the first sensor part 1070 and this increases the contact pressure between the contact points 1062 and the sensor surface 1071, thereby reinforcing the electrical contact which generates the signal output. However, it also causes the flexible arms 1061 to deflect against the axial direction of travel of the piston rod 1015, whereby elastic energy is stored therein.

In the course of the dose expelling the flexible arms 1061 remain so deflected, but when the piston rod 1015 eventually stops and the whole dose expelling system relaxes the elastic energy stored in the flexible arms 1061 is released and transferred to the sensor surface 1071 which is urged axially away from the second sensor part 1060.

The additional axial movement of the first sensor part 1070 causes an additional axial movement of the piston 1022 which in turn causes a small additional dose to be expelled. Notably, this additional dose is expelled after the piston rod 1015 has stopped its movement and will resultantly require the user to wait a little longer before removing the injection needle from the skin in order to ensure that the entire dose has been received. Furthermore, even though it is advantageous that an increased contact pressure reduces the risk of an accidental loss of contact between the contact points 1062 and the sensor surface 1071 it comes with the cost of an increased friction in the rotational interface between the first sensor part 1070 and the second sensor part 1060, which increases the risk that an angular displacement is introduced to the first sensor part 1070, thereby affecting the accuracy of the dose detection principle.

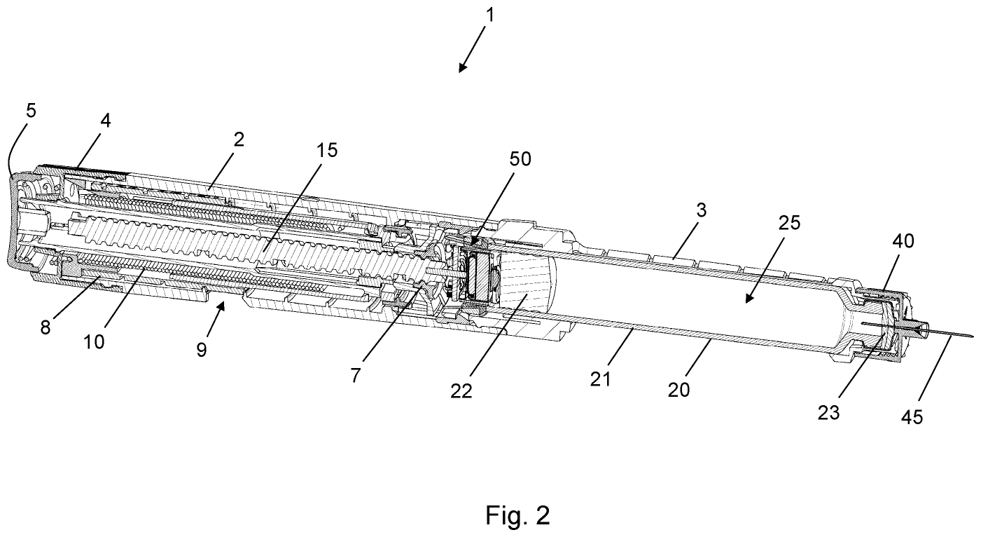

FIG. 2 is a perspective longitudinal section view of an injection device 1 having an integrated sensor module 50 according to an exemplary embodiment of the invention. The injection device 1 is of the prefilled autopen injector type, with an elongated housing 2 extending along a reference axis and accommodating a dose expelling mechanism. A cartridge holder 3, holding a cartridge 20 with an interior chamber 25 defined by a cartridge wall 21, a distal penetrable septum 23 and a proximal piston 22, is permanently fixed to the housing 2. The chamber 25 is at least substantially filled with a liquid substance (not visible). In the depicted state of the injection device 1 a needle assembly 40 is attached to a needle mount portion of the cartridge holder 3 in such a manner that an injection needle 45 has penetrated the septum 23 to establish fluid communication to the chamber 25.

A user operable dose dial 4 is arranged at a proximal end portion of the housing 2 for selective setting of a dose to be ejected from the cartridge 20. The dose dial 4 is operatively coupled with a scale drum 8 which displays a selected dose through a window 9. An injection button 5 is axially depressible to release a windable torsion spring 10. The release of the torsion spring 10 will cause a helical advancement of a piston rod 15 through a nut member 7 in the housing 2 and thereby result in an execution of a dose expelling action.

Details of the dose setting and the dose expelling mechanisms are irrelevant to the present invention and will accordingly not be provided in the present text. For an example of how such mechanisms may be constructed reference is made to WO 2015/071354, particularly p. 10, I. 21-p. 15, I. 13. What is important is that the rotational movement of the piston rod 15 during dose expelling is correlated with the prompted movement of the piston 22 through the design of the piston rod thread and the nut member 7 such that a predetermined angular displacement of the piston rod 15 relative to the housing 2 corresponds to a predetermined axial displacement of the piston 22 relative to the cartridge wall 21. This relationship may in principle be chosen arbitrarily by the manufacturer, with a view to the dimensions of the cartridge 20. In the present example a 15° angular displacement of the piston rod corresponds to a specific axial displacement of the piston 22 which results in the expelling of 1 IU of the contained substance through the injection needle 45.

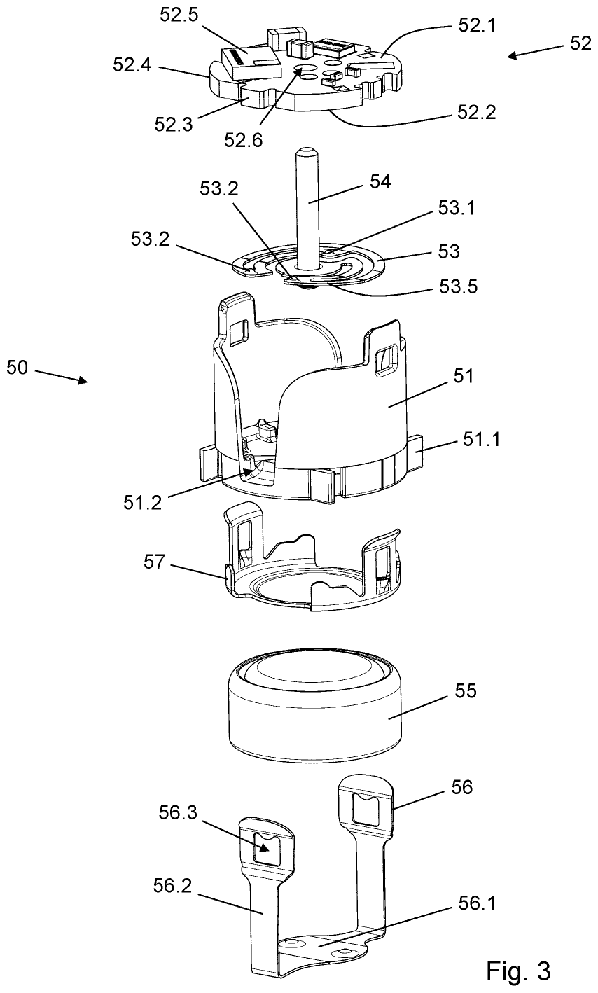

FIG. 3 is an exploded view highlighting the individual elements of the present sensor module 50. The sensor module 50 comprises a first sensor part in the form of a PCB assembly 52 with a rigid support sheet 52.4 having a proximal surface 52.1 carrying various electronic components 52.5, including a processor, and a distal surface 52.2 carrying a plurality of electrically conductive sensor areas (not visible), the configuration of which will be described below. The support sheet 52.4 has an overall circular periphery, but is provided with several notches, some of which resulting in a pair of diametrically opposite radial protrusions 52.3. Furthermore, the support sheet 52.4 has a central through-going bore 52.6.

The first sensor part is complemented by a second sensor part in the form of a wiper 53 being fixedly mounted to a piston rod connector 54 to ensure joint rotation therewith. The piston rod connector 54 extends axially through the through-going bore 52.6 and is adapted for press-fit engagement with a cavity in a distal end portion of the piston rod 15, as shown on FIG. 2. This provides for a joint movement of the piston rod 15 and the piston rod connector 54. The wiper 53 comprises one ground contact 53.1 and two code contacts 53.2 arranged on respective flexible arms 53.5 and adapted to galvanically connect with the electrically conductive sensor areas on the distal surface 52.2 of the support sheet 52.4, as described in more detail below. Notably, the ground contact 53.1 and the code contacts 53.2 are all proximally directed.

The two sensor parts, forming a rotary encoder system, are accommodated in a module housing 51 which also accommodates a power source in the form of a battery 55, a retainer 56 also functioning as a positive battery connector, and a rigid (negative) battery connector 57. The retainer 56 has a transversal support surface 56.1 for carrying the battery 55 and two axially extending opposite retainer arms 56.2. Each retainer arm 56.2 is provided with a proximal cut-out 56.3 shaped to receive one of the radial protrusions 52.3, thereby rotationally interlocking the retainer 56 and the PCB assembly 52 and axially restricting the support sheet 52.4. The module housing 51 has a pair of diametrically opposite side openings 51.2 shaped to receive the retainer arms 56.2 so as to rotationally interlock, or at least substantially rotationally interlock, the retainer 56 and the module housing 51, and a plurality of anti-rotation tabs 51.1 spaced apart along its circumference for interaction with an interior surface of the cartridge wall 21. The PCB assembly 52 is thus at least substantially rotationally locked with respect to the module housing 51, which in turn is rotationally frictionally fitted in the cartridge 20, which is rotationally fixed in the cartridge holder 3. The PCB assembly 52 is thereby at least substantially rotationally fixed with respect to the housing 2 and accordingly suitable as reference component for measuring angular displacements of the piston rod 15.

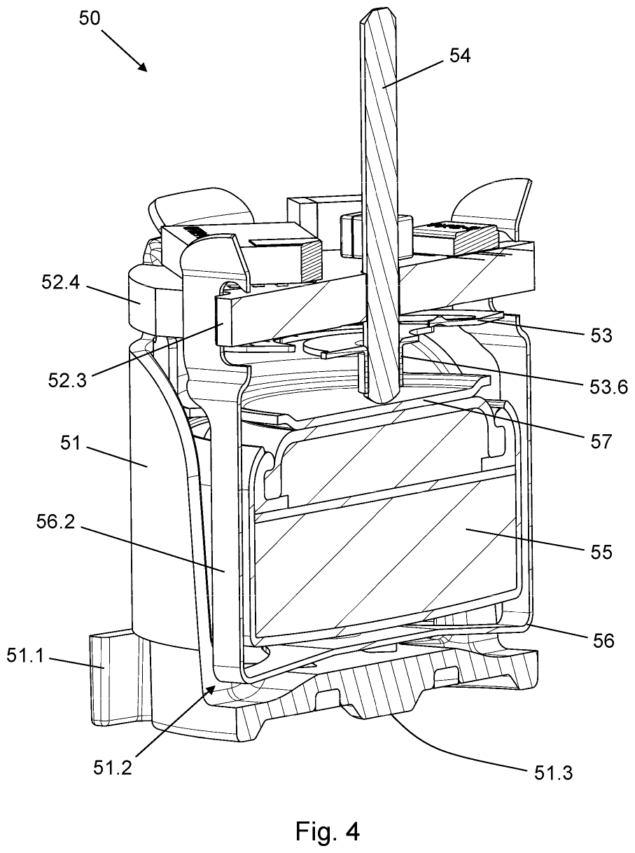

FIG. 4 is a perspective longitudinal section view of the sensor module 50 in an assembled state. As can be seen the piston rod connector 54 extends through the through-going bore 52.6 in the support sheet 52.4 and is press-fitted with a sleeve 53.6 on the wiper 53. The module housing 51 has a foot 51.3 which rests against the piston 22 (cf. FIG. 2). Furthermore, the figure shows the position of the retainer arms 56.2 in the side openings 51.2 and the arrangement of the radial protrusions 52.3 in the cut-outs 56.3. During a dose expelling action with the injection device 1 the rotation of the piston rod 15 is transferred to the piston rod connector 54 and further on to the wiper 53. The ground contact 53.1 and the code contacts 53.2 thus sweep the sensor areas of the distal surface 52.2 which remains, at least substantially, rotationally stationary due to the engagement between the radial protrusions 52.3 and the cut-outs 56.3, the fitting of the retainer arms 56.2 in the side openings 51.2, the frictional interface between the foot 51.3 and the piston 22, and the frictional interface between the anti-rotation tabs 51.1 and the cartridge wall 21.

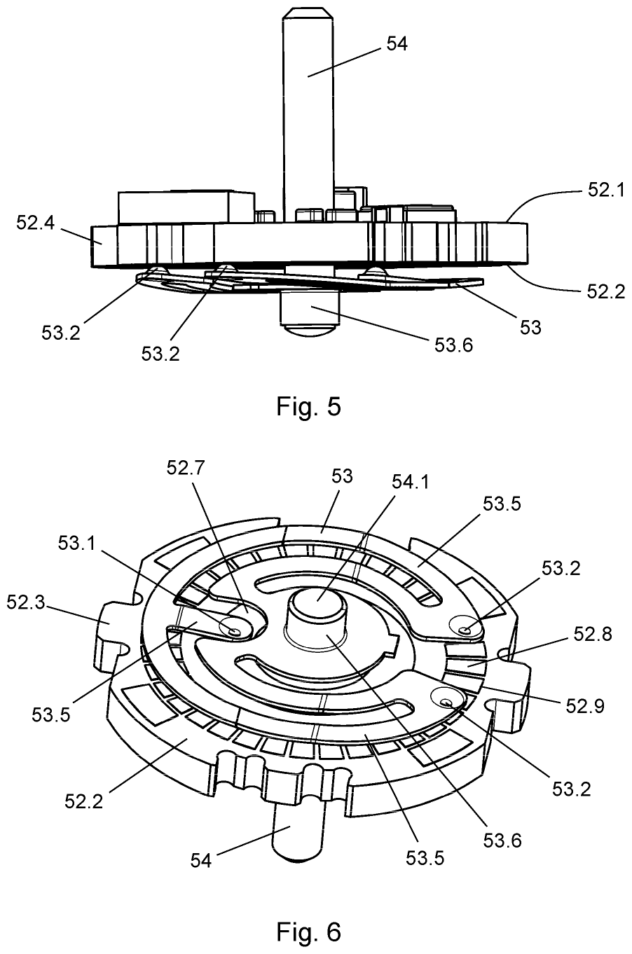

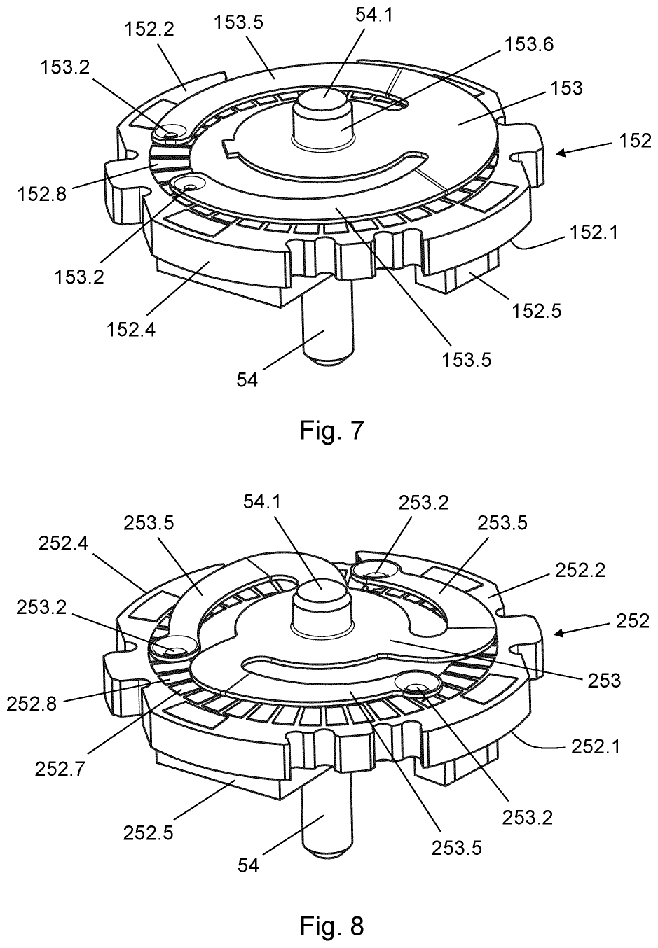

FIG. 5 is a side view of the two sensor parts showing the connection between the ground contact 53.1 and the code contacts 53.2 and the distal surface 52.2 of the support sheet 52.4, and FIG. 6 is a perspective distal view of the same. In the shown exemplary embodiment of the invention the aforementioned plurality of electrically conductive sensor areas on the distal surface 52.2 are arranged such that a single circular ground track 52.7 provides a ground connection for the ground contact 53.1 and 36 individual code fields 52.8 together constitute a code track 52.9 which the code contacts 53.2 are adapted to sweep. A secondary ground connection is provided through a spherical end 54.1 of the piston rod connector 54 contacting the (negative) battery connector 57. The secondary ground connection may be relevant to stabilise the signal output in case the dynamics of the dose expelling mechanism generates vibrations in the sensor module 50.

As the piston rod connector 54 rotates jointly with the piston rod 15 during a dose expelling action the two code contacts 53.2, which are circumferentially separated by 45°, respectively sweep the code track 52.9, generating signals representative of the angular position of the wiper 53 as different code fields 52.8 get connected to ground. The two sensor parts output a 4-bit Gray code, i.e. eight different codes which for a 360° rotation of the wiper 53 are repeated nine times, giving 72 distinguishing codes. This output thus forms the basis for an estimation, by one or more of the electronic components 52.5 including the processor, of the total angular displacement of the piston rod 15 during a dose expelling action, and thereby for an estimation of the expelled dose.

For galvanic sensors like the herein described it is crucial that the contact pressure on each physical contact is sufficiently high to ensure a stable signal. This prerequisite is met by the design of the present sensor module 50, where the combination of the flexible arms 53.5 and the sleeve 53.6 and the restricted axial play of the radial protrusions 52.3 in the cut-outs 56.3 enables an arrangement of the wiper 53 on the piston rod connector 54 relative to the support sheet 52.4 which provide a spring reinforced contact between the ground contact 53.1 and the ground track 52.7 as well as between the respective code contacts 53.2 and the code track 52.9. However, importantly, the fact that the wiper 53 is positioned distally of the support sheet 52.4 such that the flexible arms 53.5 are deflected distally and the respective ground and code contacts 53.1, 53.2 thereby provide proximally directed forces to the support sheet 52.4 is advantageous because during a dose expelling action when the piston rod connector 54 applies an axially directed force to the battery connector 57 this will not result in a further deflection of the flexible arms 53.5 as the wiper 53 is not pressed against the support sheet 52.4, i.e. no additional elastic energy is stored in the flexible arms 53.5 which needs to be released during the subsequent relaxation of the dose expelling system, and the problem of prolonged dose expelling is thus solved.

Furthermore, since the wiper 53 is not being pressed against the support sheet 52.4 as a result of the advancing piston rod connector 54 the contact pressure in the respective ground contact 53.1/ground track 52.7 and code contact 53.2/code track 52.9 interfaces is not increased during dose delivery. The friction in the rotational interface between the two sensor parts is therefore also not increased, which means that the torque applied by the wiper 53 to the support sheet 52.4 is not increased. The risk of angular displacement of the support sheet 52.4 against the rotation prevention mechanism provided by the interaction between the anti-rotation tabs 51.1 and the cartridge wall 21 is resultantly reduced compared to a solution, e.g. like the one shown in FIG. 1, where the flexible arms exhibit further deflection during piston rod advancement.

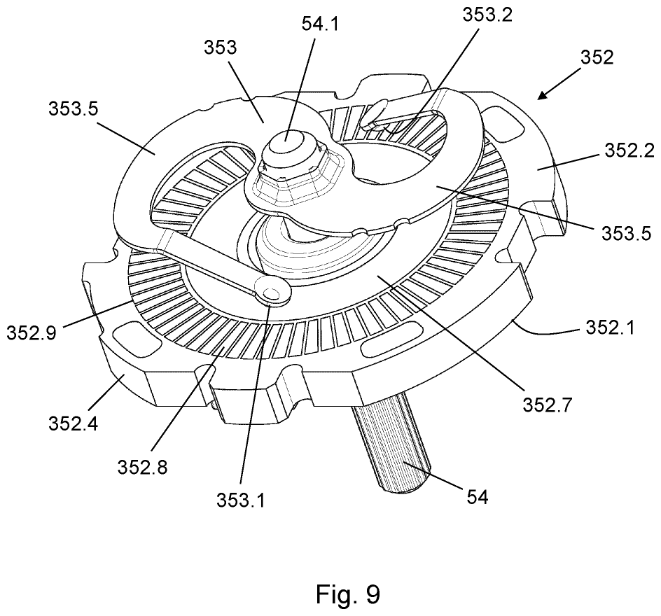

FIG. 7 is a perspective distal view of two sensor parts of an alternative rotary encoder system used in a sensor module according to another embodiment of the invention. The sensor parts comprise a wiper 153 and a PCB assembly 152 held in mutual position by the piston rod connector 54 in a manner similar to that disclosed in connection with the previous embodiment. The geometrical configuration of the PCB assembly 152 as well as its interaction with other components of the sensor module is identical to that of the formerly described PCB assembly 52. Particularly, the PCB assembly 152 comprises a rigid support sheet 152.4 having a proximal surface 152.1 which carries various electronic components 152.5, including a processor, and a distal surface 152.2 on which is disposed a plurality of electrically conductive code fields 152.8 arranged side by side to thereby provide a circular code track. However, contrary to the former embodiment the distal surface 152.2 does not comprise a dedicated ground track. Instead, the ground connection is supplied via the spherical end 54.1 of the piston rod connector 54 being in contact with the (negative) battery connector 57, similarly to the above described.

The wiper 153 comprises a sleeve 153.6 press-fitted onto the piston rod connector 54, to ensure joint rotation of the piston rod 15 and the wiper 153, and two code contacts 153.2, each arranged at an end portion of a flexible arm 153.5 capable of axial deflection. The code contacts 153.2 are angularly separated by 45° and will when rotated relative to the distal surface 152.2 respectively sweep the code fields 152.8 and produce a 4-bit Gray code, similarly to the previous embodiment. The fact that only two wiper contacts sweep the distal surface 152.2 provides for a reduced internal friction and therefore a reduced torque between the two sensor parts, compared to three sweeping contacts. Hence, the risk of angular displacement of the PCB assembly 152 against the rotation prevention mechanism provided by the interaction between the anti-rotation tabs 51.1 and the cartridge wall 21 is reduced even further, while the advantageous containment of the forces from the flexible arms 153.5 between the PCB assembly 152 and the battery 55 is still obtained, eliminating the prolonged dose expelling problem.

FIG. 8 is a perspective distal view of two sensor parts of another alternative rotary encoder system used in a sensor module according to a third embodiment of the invention. Similarly to the previous embodiments the sensor parts comprise a wiper 253 and a PCB assembly 252 held in mutual position by the piston rod connector 54. The geometrical configuration of the PCB assembly 252 as well as its interaction with other components of the sensor module is identical to that of the formerly described PCB assembly 52. Particularly, the PCB assembly 252 comprises a rigid support sheet 252.4 having a proximal surface 252.1 which carries various electronic components 252.5, including a processor, and a distal surface 252.2 on which is disposed a plurality of electrically conductive sensor areas.

However, contrary to the former embodiments the distal surface 252.2 carries 40 electrically conductive sensor areas arranged in a circular track pattern where every other sensor area constitutes a ground field 252.7 and every other sensor area constitutes a code field 252.8. A secondary ground connection is supplied via the spherical end 54.1 of the piston rod connector 54 being in contact with the (negative) battery connector 57, as described above in connection with the first embodiment of the invention.

The wiper 253 is attached to the piston rod connector 54 and is adapted to sweep the 40 electrically conductive sensor areas as the piston rod 15 rotates during a dose expelling action (as described above). The wiper 253 has three flexible arms 253.5, each terminating in a contact point 253.2 which is adapted to galvanically connect with a ground field 252.7 or a code field 252.8, depending on the angular position of the wiper 253 relative to the PCB assembly 252. The three contact points 253.2 are separated 120° from each other such that one contact point 253.2 is always connected to a ground field 252.7 and two contact points 253.2 are always connected to a code field 253.8. The two sensor parts output a 4-bit Gray code and offer a higher resolution than the former two embodiments of the invention, enabling an even more accurate estimation of the total relative angular displacement between the PCB assembly 252 and the wiper 253, and thereby of the total angular displacement of the piston rod 15 relative to the housing 2, during a dose expelling event.

FIG. 9 is a perspective distal view of two sensor parts of yet another alternative rotary encoder system used in a sensor module according to a fourth embodiment of the invention. Like in the previous embodiments the sensor parts comprise a wiper 353 and a PCB assembly 352 held in mutual position by the piston rod connector 54. The geometrical configuration of the PCB assembly 352 as well as its interaction with other components of the sensor module correspond to that of the formerly described PCB assembly 52. Particularly, the PCB assembly 352 comprises a rigid support sheet 352.4 having a proximal surface 352.1 which carries various electronic components (not visible), including a processor, and a distal surface 352.2 on which is disposed a plurality of electrically conductive sensor areas. The plurality of electrically conductive sensor areas comprises a circular ground track 352.7 and a circular code track 352.9 formed of 72 individual code fields 352.8 which are arranged side by side.

The wiper 353 is press-fitted onto the piston rod connector 54, to ensure joint rotation with the piston rod 15, and comprises a code contact 353.2 and a diametrically opposite ground contact 353.1, each contact arranged at an end portion of a flexible arm 353.5 capable of axial deflection. During a dose expelling action when the wiper 353 rotates relative to the PCB assembly 352 the code contact 353.2 will sweep at least a subset of the code fields 352.8 while the ground contact 353.1 will sweep at least a subset of the ground track 352.7. This produces a number of signal shifts which can be correlated with a particular angular displacement of the piston rod 15 relative to the housing 2 and thus used to estimate the size of the expelled dose.

Again, the fact that only two wiper contacts sweep the distal surface 352.2 provides for a reduced internal friction and therefore a reduced torque between the two sensor parts, compared to three sweeping contacts. Hence, the risk of angular displacement of the PCB assembly 352 against the rotation prevention mechanism provided by the interaction between the anti-rotation tabs 51.1 and the cartridge wall 21 is reduced even further, while the advantageous containment of the forces from the flexible arms 353.5 between the PCB assembly 352 and the battery 55 is still obtained, eliminating the prolonged dose expelling problem

In a variation of the above sequential encoder, the ground track 352.7 and the flexible arm 353.5 carrying the ground contact 353.1 could be omitted and ground connection could be provided solely by the spherical end 54.1 of the piston rod connector 54 contacting the (negative) battery connector 57. This would reduce the internal friction even further as only one wiper contact would sweep the distal surface 352.2. In order to enhance the structural stability of this alternative wiper, it could be considered to introduce an arm to counterbalance the flexible arm 353.5 carrying the code contact 353.2.

1. A sensor module adapted to be arranged in a cartridge based drug delivery device between a rotatable piston rod and a cartridge piston, the sensor module extending along a reference axis from a proximal module portion adapted to interface with the piston rod to a distal module portion adapted to interface with the cartridge piston and comprising:
a module housing, and
a powered rotary encoder system comprising:
a first sensor structure adapted to be at least substantially rotationally locked with respect to the cartridge piston and comprising a transversal sensor surface axially restricted with respect to the module housing,
a second sensor structure adapted to be rotationally locked with respect to the piston rod and comprising one or more flexibly supported and axially deflectable contact members, the first sensor structure and the second sensor structure being capable of undergoing relative rotational motion about the reference axis, whereby the one or more contact members sweep the transversal sensor surface, and
a processor adapted to determine a relative angular displacement between the first sensor structure and the second sensor structure from signals generated as the one or more contact members sweep the transversal sensor surface,
wherein the one or more contact members are positioned distally of the transversal sensor surface and adapted to apply a proximally directed force thereto.

2. A sensor module according to claim 1, wherein
the transversal sensor surface comprises a plurality of electrically conductive sensor areas arranged in a pattern, and
the one or more contact members are adapted to sweep at least a subset of the plurality of electrically conductive sensor areas as the first sensor structure and the second sensor structure undergo relative rotation, thereby alternately connecting and disconnecting different sensor areas, a current connection being indicative of a current relative angular position of the first sensor structure and the second sensor structure.

3. A sensor module according to claim 1, wherein the transversal sensor surface is a distal surface of a rigid support sheet which extends perpendicularly to the reference axis within the module housing.

4. A sensor module according to claim 3, wherein the proximal module portion comprises an axial pin member comprising a proximal pin end portion and a distal pin end portion, and
wherein
the rigid support sheet has a central through-going bore,
the axial pin member extends through the through-going bore,
the second sensor structure is rotationally interlocked with the distal pin end portion, and
the proximal pin end portion is configured for rotational interlocking engagement with a distal end portion of the piston rod.

5. A sensor module according to claim 3, wherein the rigid support sheet further comprises a proximal surface carrying the processor.

6. A sensor module according to claim 1, further comprising anti-rotation structure adapted to interface with an interior wall portion of a cartridge in the cartridge based drug delivery device to impede relative angular displacement between the module housing and the cartridge.

7. A sensor module according to claim 2, wherein the plurality of electrically conductive sensor areas are arranged to form a first circular track and a second circular track, the first circular track being a code track and the second circular track being a ground track, and
wherein the one or more contact members constitute one code contact member adapted to sweep the first circular track and one ground contact member adapted to sweep the second circular track.

8. A sensor module according to claim 2, wherein the plurality of electrically conductive sensor areas are arranged to form a first circular track and
a second circular track, the first circular track being a code track and the second circular track being a ground track, and
wherein the one or more contact members constitute two code contact members adapted to sweep the first circular track and one ground contact member adapted to sweep the second circular track.

9. A sensor module according to claim 8, wherein the first circular track comprises 36 evenly distributed code fields, and the code contact members exhibit a 45° angular separation.

10. A sensor module according to claim 2, wherein the plurality of electrically conductive sensor areas form a single circular track comprising 40 evenly distributed fields where every other field is a code field and every other field is a ground field, and
wherein the one or more contact members constitute three contact members exhibiting a 120° angular separation from each other.

11. A sensor module according to claim 7, further comprising a battery arranged in the module housing distally of the transversal sensor surface,
wherein the proximal module portion comprises an axial pin member comprising a proximal pin end portion configured for rotational interlocking engagement with a distal end portion of the piston rod and a distal pin end portion with which the second sensor structure is rotationally interlocked,
wherein the transversal sensor surface is a distal surface of a rigid support sheet which extends perpendicularly to the reference axis within the module housing and has a central through-going bore, and
wherein the axial pin member extends through the through-going bore and the distal pin end portion comprises a contact surface which abuts the battery, connecting electrically to a negative battery terminal.

12. A sensor module according to claim 2, further comprising a battery arranged in the module housing distally of the transversal sensor surface,
wherein the plurality of electrically conductive sensor areas form a single circular track comprising 36 evenly distributed code fields and the one or more contact members constitute two code contact members exhibiting a 45° angular separation,
wherein the proximal module portion comprises an axial pin member comprising a proximal pin end portion configured for rotational interlocking engagement with a distal end portion of the piston rod and a distal pin end portion with which the second sensor structure is rotationally interlocked,
wherein the transversal sensor surface is a distal surface of a rigid support sheet which extends perpendicularly to the reference axis within the module housing and has a central through-going bore, and
wherein the axial pin member extends through the through-going bore and the distal pin end portion comprises a contact surface which abuts the battery, connecting electrically to a negative battery terminal.

13. A sensor module according to claim 1 in combination with a drug delivery device comprising:
a housing accommodating a dose expelling mechanism comprising a piston rod, and
a cartridge rotationally fixed with respect to the housing, the cartridge comprising a drug chamber, sealed distally by a self-sealing septum and proximally by a cartridge piston,
wherein the proximal module portion is rotationally fixed to the piston rod and the distal module portion abuts the cartridge piston.

14. A sensor module and drug delivery device according to claim 13, wherein the proximal module portion is friction-fitted to an indentation in the piston rod.
